(19) United States
(12) Patent Application Publication
(10) Pub. No.: US 2005/0131650 A1
(43) Pub. Date: Jun. 16, 2005

(54) Method and system for interaction analysis

(75) Inventors: Karl Andersson, Uppsala (SE); Peter Borg, Uppsala (SE); Annica Onell, Uppsala (SE)

Correspondence Address: SEED INTELLECTUAL PROPERTY LAW GROUP PLLC, 701 FIFTH AVE, SUITE 6300, SEATTLE, WA, 98104-7092, US

(73) Assignee: Biacore AB, Uppsala (SE)

(21) Appl. No.: 10/945,567

(22) Filed: Sep. 20, 2004

Related U.S. Application Data
(60) Provisional application No. 60/505,914, filed on Sep. 24, 2003.

(30) Foreign Application Priority Data
Sep. 24, 2003 (SE) 0302525-1

(51) Int. Cl. G06F 19/00; G01N 33/48; G01N 33/50; G01N 31/00

(52) U.S. Cl. 702/19; 702/22

(57) ABSTRACT

The invention relates to a computer-implemented method for determining at least one kinetic parameter for the interaction of an analyte in solution with an immobilized ligand from a data set comprising a plurality of different binding curves, each of which represents the progress of the interaction of the analyte with the ligand with time, comprising the steps of: a) performing at least one fit of the whole data set or subsets thereof to a predetermined kinetic model for the interaction; b) based on the result of the fit or fits performed in step a), identifying and excluding binding curves of unacceptable quality; c) performing a final fit to the remaining data set; and d) obtaining therefrom the kinetic parameter or parameters. The invention also relates to an analytical system for carrying out the method, as well as a computer program, computer program product and computer system for performing the method.

### CROSS-REFERENCE TO RELATED APPLICATION

This application claims the benefit of U.S. Provisional Patent Application No. 60/505,914, filed Sep. 24, 2003, and also claims priority from Swedish Patent Application No. 0302525-1, filed Sep. 24, 2003; both of which applications are incorporated here by reference in their entireties.

### BACKGROUND OF THE INVENTION

1. Field of the Invention

The present invention relates to a method of analysing molecular binding interactions at a sensing surface, and more particularly to an at least partially automated method for determining kinetic parameters from the resulting data describing the molecular interactions. The invention also relates to an analytical system including means for such automated kinetic evaluation as well as to a computer program for performing the method, a computer program product comprising program code means for performing the method, and a computer system containing the program.

2. Description of the Related Art

Analytical sensor systems that can monitor interactions between molecules, such as biomolecules, in real time are gaining increasing interest. These systems are often based on optical biosensors and usually referred to as interaction analysis sensors or biospecific interaction analysis sensors. A representative such biosensor system is the BIACORE® instrumentation sold by Biacore AB (Uppsala, Sweden), which uses surface plasmon resonance (SPR) for detecting interactions between molecules in a sample and molecular structures immobilized on a sensing surface. As sample is passed over the sensor surface, the progress of binding directly reflects the rate at which the interaction occurs. Injection of sample is followed by a buffer flow during which the detector response reflects the rate of dissociation of the complex on the surface. A typical output from the BIACORE® system is a graph or curve describing the progress of the molecular interaction with time. This binding curve, which is usually displayed on a computer screen, is often referred to as a “sensorgram”.

With the BIACORE® system (and analogous sensor systems) it is thus possible to determine in real time without the use of labeling, and often without purification of the substances involved, not only the presence and concentration of a particular molecule in a sample, but also additional interaction parameters, including kinetic rate constants for binding and dissociation in the molecular interaction. The association and dissociation rate constants can be obtained by fitting the resulting kinetic data to mathematical descriptions of interaction models in the form of differential equations. While such kinetic analysis is usually assisted by dedicated software, intervention by the operator is required during the iterative curve fitting process to inter alia identify and exclude binding curves which give rise to a bad fit, for example, due to assay-related faults, such as, for example, the presence of particles in a sample. Binding curves of unacceptable quality due to instrument-related faults, such as, e.g., air spikes caused by air bubbles in the fluid flow, are normally discarded in a curve quality control performed prior to the kinetic analysis.

It is readily seen that the current trend towards systems with ever increasing throughput and information density in the analyses performed puts a more and more heavy burden on the operator. To reduce the work by the operator to some extent, an automated curve quality control procedure is disclosed in U.S. patent application publication U.S. 2004/0002167 A1. There is, however, still a need for means that facilitate the kinetic evaluation of molecular interaction data obtained in biosensor systems, especially where large sets of interaction data, such as sensorgrams, are produced.

### BRIEF SUMMARY OF THE INVENTION

It is an object of the present invention to improve the kinetic evaluation of molecular interaction data, such as real-time biosensor data.

Therefore, in one aspect, the present invention provides a computer-implemented method of determining at least one kinetic parameter for the interaction of an analyte in solution with an immobilized ligand from a data set comprising a plurality of different binding curves, each of which represents the progress of the interaction of the analyte with the ligand with time, which method comprises the steps of:
a) performing at least one fit of the whole data set or subsets thereof to a predetermined kinetic model for the interaction;
b) based on the result of the fit or fits performed in step a), identifying and excluding binding curves of unacceptable quality;
c) performing a final fit to the remaining data set; and
d) obtaining therefrom the kinetic parameter or parameters.

Optionally, steps a) and b) may be iterated until no binding curves with unacceptable quality are identified.

If the remaining data set after step b) is identical to a data set (the whole data set or a data subset) to which a fit has been made in step a), step c) may be omitted and the kinetic parameter(s) may be obtained from the fit in step a) (when no binding curves are excluded, the “remaining” data set in step c) is, of course, identical to the whole data set in step a)).

The terms “analyte” and “ligand” as used herein are to be interpreted in a broad sense. Basically, ligand means an entity that has a known or unknown affinity for a given analyte. The ligand may be a naturally occurring species or one that has been synthesized. The ligand is usually a biomolecule.

Common analytes include biomolecules (such as proteins, peptides, DNA, RNA, and the like), chemicals purified from extracts of biological material (e.g., plant extracts), synthesized chemicals (including small molecules), cells and viruses.

In another aspect, the present invention provides an analytical system for studying molecular interactions, which comprises data processing means for performing the above method.

In still another aspect, the present invention provides a computer program comprising program code means for performing the method.

In yet another aspect, the present invention provides a computer program product comprising program code means stored on a computer readable medium or carried on an electrical or optical signal for performing the method.

In still another aspect, the present invention provides a computer system containing a computer program comprising program code means for performing the method.

These and other aspects of this invention will be evident upon reference to the accompanying drawings and the following detailed description.

### BRIEF DESCRIPTION OF THE DRAWINGS

FIG. 1 is a schematic side view of a biosensor system based on SPR.

FIG. 2 is a representative sensorgram where the binding curve has visible association and dissociation phases.

FIG. 3 is a flow chart showing an exemplary algorithm for carrying of the method of the present invention.

FIG. 4 is a flow chart showing another exemplary algorithm for carrying of the method of the present invention.

FIG. 5 shows (A) overlay sensorgrams for the interaction of a drug (CBSA) with a sensing surface, (B) the corresponding sensorgrams together with fitted binding curves and indicated outlier sensorgrams, and (C) the corresponding sensorgrams together with fitted binding curves after exclusion of outliers.

FIG. 6 shows (A) overlay sensorgrams for the interaction of a drug (indapamide) with a sensing surface, (B) the corresponding sensorgrams together with fitted binding curves and indicated outlier sensorgrams, and (C) the corresponding sensorgrams together with fitted binding curves after exclusion of outliers.

FIG. 7 shows (A) overlay sensorgrams for the interaction of a drug (furosemide) with a sensing surface, (B) the corresponding sensorgrams together with fitted binding curves and indicated outlier sensorgrams, and (C) the corresponding sensorgrams together with fitted binding curves after exclusion of outliers.

### DETAILED DESCRIPTION OF THE INVENTION

Unless defined otherwise, all technical and scientific terms used herein have the same meaning as commonly understood by a person skilled in the art related to this invention. Also, the singular forms “a”, “an”, and “the” are meant to include plural reference unless it is stated otherwise.

As mentioned above, the present invention relates to analytical sensor methods, particularly biosensor based methods, where molecular interactions are studied and the results are presented in real time, as the interactions progress, in the form of detection curves, often called sensorgrams.

While biosensors are typically based on label-free techniques, detecting, e.g., a change in mass, refractive index or thickness for the immobilized layer, there are also sensors relying on some kind of labelling. Typical sensor detection techniques include, but are not limited to, mass detection methods, such as optical, thermo-optical and piezoelectric or acoustic wave methods (including, e.g., surface acoustic wave (SAW) and quartz crystal microbalance (QCM) methods), and electrochemical methods, such as potentiometric, conductometric, amperometric and capacitance/impedance methods. With regard to optical detection methods, representative methods include those that detect mass surface concentration, such as reflection-optical methods, including both external and internal reflection methods, angle, wavelength, polarization, or phase resolved, for example evanescent wave ellipsometry and evanescent wave spectroscopy (EWS, or Internal Reflection Spectroscopy), both of which may include evanescent field enhancement via surface plasmon resonance (SPR), Brewster angle refractometry, critical angle refractometry, frustrated total reflection (FTR), scattered total internal reflection (STIR), which may include scatter enhancing labels, optical wave guide sensors, external reflection imaging, evanescent wave-based imaging such as critical angle resolved imaging, Brewster angle resolved imaging, SPR-angle resolved imaging, and the like. Further, photometric and imaging/microscopy methods, “per se” or combined with reflection methods, based on for example surface enhanced Raman spectroscopy (SERS), surface enhanced resonance Raman spectroscopy (SERRS), evanescent wave fluorescence (TIRF) and phosphorescence may be mentioned, as well as waveguide interferometers, waveguide leaking mode spectroscopy, reflective interference spectroscopy (RIfs), transmission interferometry, holographic spectroscopy, and atomic force microscopy (AFR).

Commercially available biosensors include the BIACORE® system instruments, marketed by Biacore AB, Uppsala, Sweden, which are based on surface plasmon resonance (SPR) and permit monitoring of surface binding interactions in real time berween a bound ligand and an analyte of interest.

The phenomenon of SPR is well known, suffice it to say that SPR arises when light is reflected under certain conditions at the interface between two media of different refractive indices, and the interface is coated by a metal film, typically silver or gold. In the BIACORE® instruments, the media are the sample and the glass of a sensor chip that is contacted with the sample by a microfluidic flow system. The metal film is a thin layer of gold on the chip surface. SPR causes a reduction in the intensity of the reflected light at a specific angle of reflection. This angle of minimum reflected light intensity varies with the refractive index close to the surface on the side opposite from the reflected light, in the BIACORE® system the sample side.

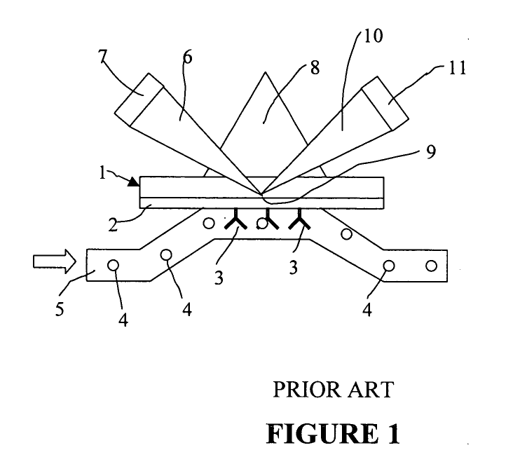

A schematic illustration of the BIACORE® system is shown in FIG. 1. Sensor chip 1 has a gold film 2 supporting capturing molecules 3, e.g., antibodies, exposed to a sample flow with analytes 4 (e.g., an antigen) through a flow channel 5. Monochromatic p-polarised light 6 from a light source 7 (LED) is coupled by a prism 8 to the glass/metal interface 9 where the light is totally reflected. The intensity of the reflected light beam 10 is detected by an optical detection unit (photodetector array) 11.

A detailed discussion of the technical aspects of the BIACORE instrument and the phenomenon of SPR may be found in U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used in connection with the BIACORE® instrument may be found in U.S. Pat. No. 5,492,840. The full disclosures of the above-mentioned U.S. patents are incorporated by reference herein.

When molecules in the sample bind to the capturing molecules on the sensor chip surface, the concentration, and therefore the refractive index at the surface changes and an SPR response is detected. Plotting the response against time during the course of an interaction will provide a quantitative measure of the progress of the interaction. Such a plot is usually called a sensorgram. In the BIACORE® system, the SPR response values are expressed in resonance units (RU). One RU represents a change of 0.0001° in the angle of minimum reflected light intensity, which for most proteins and other biomolecules correspond to a change in concentration of about 1 pg/mm2 on the sensor surface. As sample containing an analyte contacts the sensor surface, the ligand bound to the sensor surface interacts with the analyte in a step referred to as “association.” This step is indicated on the sensorgram by an increase in RU as the sample is initially brought into contact with the sensor surface. Conversely, “dissociation” normally occurs when the sample flow is replaced by, for example, a buffer flow. This step is indicated on the sensorgram by a drop in RU over time as analyte dissociates from the surface-bound ligand.

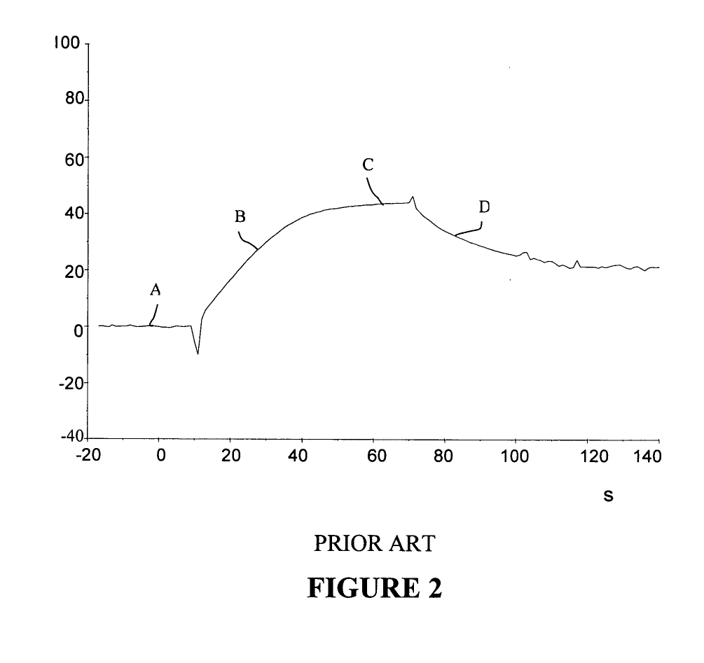

A representative sensorgram (binding curve) for a reversible interaction at the sensor chip surface is presented in FIG. 2, the sensing surface having an immobilized capturing molecule, for example an antibody, interacting with analyte in a sample. The y-axis indicates the response (here in resonance units, RU) and the x-axis indicates the time (here in seconds). Initially, buffer is passed over the sensing surface giving the baseline response A in the sensorgram. During sample injection, an increase in signal is observed due to binding of the analyte. This part B of the binding curve is usually referred to as the “association phase”. Eventually, a steady state condition is reached where the resonance signal plateaus at C. At the end of sample injection, the sample is replaced with a continuous flow of buffer and a decrease in signal reflects the dissociation, or release, of analyte from the surface. This part D of the binding curve is usually referred to as the “dissociation phase”. The analysis is usually ended by a regeneration step (not shown in FIG. 2) where a solution capable of removing bound analyte from the surface, while (ideally) maintaining the activity of the ligand, is injected over the sensor surface. Injection of buffer restores the baseline A and the surface is then ready for a new analysis.

As will be explained in more detail below, the profiles of the association and dissociation phases B and D, respectively, provide valuable information regarding the interaction kinetics, and the height of the resonance signal represents surface concentration (i.e., the response resulting from an interaction is related to the change in mass concentration on the surface).

The detection curves, or sensorgrams, produced by biosensor systems based on other detection principles mentioned above will have a similar appearance.

Assume a reversible reaction (which is not diffusion or mass transfer limited) between an analyte A and a surface-bound (immobilized) capturing molecule, or ligand, B (first order kinetics):
A+B [symbol] AB

This model (usually referred to as the Langmuir model), which assumes that the analyte (A) is both monovalent and homogenous, that the ligand (B) is homogenous, and that all binding events are independent, is in fact applicable in the vast majority of cases.

The rate of change in surface concentration of A during analyte injection is

$$\frac{ⅆ Γ}{ⅆ t} = {k}_{ass} ( {Γ}_{max} - Γ ) C - {k}_{diss} Γ \qquad ( 1 )$$

where Γ is the concentration of bound analyte, Γₘₐₓ is the maximum binding capacity of the surface, kₐₛₛ is the association rate constant, k_{diss} is the dissociation rate constant, and C is the bulk analyte concentration. Rearrangement of the equation gives:

$$\frac{ⅆ Γ}{ⅆ t} = {k}_{ass} C {Γ}_{max} - ( {k}_{ass} C + {k}_{diss} ) Γ \qquad ( 2 )$$

If all concentrations are measured in the same units, the equation may be rewritten as:

$$\frac{ⅆ R}{ⅆ t} = {k}_{ass} C {R}_{max} - ( {k}_{ass} C + {k}_{diss} ) R \qquad ( 3 )$$

where R is the response in RU. In integrated form, the equation is:

$$R = \frac{{k}_{ass} {CR}_{max}}{{k}_{ass} C + {k}_{diss}} ( 1 - {ⅇ}^{- ( {k}_{ass} C + {k}_{diss} ) t} ) \qquad ( 4 )$$

Now, according to equation (3), if dR/dt is plotted against the bound analyte concentration R, the slope is kₐₛₛC+k_{diss} and the vertical intercept is kₐₛₛRₘₐₓC. If the bulk concentration C is known and Rₘₐₓ has been determined (e.g., by saturating the surface with a large excess of analyte), the association rate constant kₐₛₛ and the dissociation rate constant k_{diss} can be calculated. A more convenient method is, however, fitting of the integrated function (4), or numerical calculation and fitting of the differential Equation (3), preferably by means of a computer program as will be described below.

The rate of dissociation can be expressed as:

$$\frac{ⅆ R}{ⅆ t} = - {k}_{diss} R \qquad ( 5 )$$

and in integrated form:
R=R₀e^{-k}^{_{diss}}^{-k}_{_{diss}}^{l} (6)
where R₀ is the response at the beginning of the dissociation phase.

Alternatively, equation (6) may be linearized:
ln [R/R₀]=−k_{diss}_{^{·t}} (7)
and a plot of ln [R/R₀] vs t will produce a straight line with the slope=−k_{diss}.

Affinity is expressed by the association constant K_{A}=kₐₛₛ/k_{diss} or the dissociation constant K_{D}=k_{diss}/kₐₛₛ.

Analysis of kinetic data produced by the Biacore® instruments is usually performed using the dedicated BIAevaluation software (supplied by Biacore AB, Uppsala, Sweden) using numerical integration to calculate the differential rate equations and non-linear regression to fit the kinetic parameters. Basically, such software-assisted data analysis is performed as follows. After subtracting background noises, an attempt is made to fit the above-mentioned simple 1:1 Langmuir binding model as expressed by equations (4) and (6) above to the measurement data. Usually the binding model is fitted simultaneously to multiple binding curves obtained with different analyte concentrations C (or with different levels of surface derivatization Rₘₐₓ). Based on the sensorgram data such a “global” fitting establishes whether a single global kₐₛₛ or k_{diss} will provide a good fit to all the data. The results of the completed fit is presented to the operator graphically, displaying the fitted curves overlaid on the original sensorgram curves. The closeness of the fit is also presented by the chi-squared (χ²) value, a standard statistical measure. For a good fitting, the chi-squared value is in the same magnitude as the noise in RU². Optionally, “residual plots” are also provided which give a graphical indication of how the experimental data deviate from the fitted curve showing the difference between the experimental and fitted data for each curve. The operator then decides if the fit is good enough. If not, the sensorgram or sensorgrams exhibiting the poorest fit are excluded and the fitting procedure is run again with the reduced set of sensorgrams. This procedure is repeated until the fit is satisfactory.

Sometimes, the above-mentioned 1:1 binding reaction model will not be valid, which requires the data set to be reanalysed using one or more other reaction models. Such alternative models may include, for example, a one to one reaction influenced by mass transfer, two parallel independent one to one reactions, two competing reactions, and a two state reaction. Parallel reactions can occur when the immobilized ligand is heterogeneous, whereas a heterogenous analyte may give rise to competing reactions. A two state reaction indicates a conformation change that gradually leads to a more stable complex between ligand and analyte. For differential rate equations reflecting these alternative reaction models, it may be referred to, for example, Karlsson, R., and Fält, A., J. Immunol. Methods 200 (1997) 121-133 (the disclosure of which is incorporated by reference herein). For a more comprehensive description of curve fitting with regard to the BIACORE® system, it may be referred to the BIAevaluation Software Handbook (Biacore AB, Uppsala, Sweden) (the disclosure of which is incorporated by reference herein).

While the above described computer-assisted fitting procedure is quite manageable to the operator for a moderate number of sensorgrams or individual analyte-ligand interactions, such as, e.g., about 100 sensorgrams or 5 analyte-ligand interactions, it is readily seen that for a larger number of sensorgrams, say about 1000 sensorgrams or 50 analyte-ligand interactions, the determination of kinetic constants will be a very tedious and time-consuming task. In view of the current trend towards high throughput biosensor systems capable of producing large sets of sensorgrams in a relatively short time, a more automated binding data evaluation process is therefore required. According to the present invention, there is provided such a kinetic analysis method, which facilitates the work of the operator substantially and permits the kinetic evaluation of large numbers of sensorgrams in a short time.

Basically, the method of the invention provides for an automated curve fitting and assessment procedure that, without intermediate decisions by the operator, excludes bad sensorgrams, reiterates the fit on the reduced data set, and presents the calculated kinetic constants to the operator, preferably together with information on the goodness of the fit. For a set of binding curve data, such as the interaction between an analyte at different concentrations with an immobilized ligand, the method comprises the following steps:
a) performing at least one fit on the whole or parts of the data set,
b) from the result of step a), identifying and excluding unacceptable binding curves from the data set,
c) performing a final fit on the remaining binding curves of the data set, and
d) presenting the results.

Steps a) and b) may be iterated until no more binding curves with unacceptable quality are identified.

If more than one data set is handled simultaneously, the results from step c) are preferably presented in order of quality.

It is understood that in some cases, the fit or one of the fits performed in step a) may be acceptable, and no final fit will, of course, then be necessary. This is, for example, the case when a fit has been made to the whole data set and the result is acceptable without exclusion of any binding curves, or when a binding curve or curves have been excluded but the remaining data set is identical to a data subset to which a fit has already been made in step a).

It is to be noted that the term “binding curve” as used herein is to be interpreted in a broad sense. Thus, while FIG. 2 shows a response curve as obtained when monitoring the temporary interaction of an analyte at a defined concentration with an immobilized ligand, “binding curve” may refer not only to the whole response curve but also to only a part thereof, such as, e.g., the association part (or a part thereof) or the dissociation part (or a part thereof). Also, in, e.g., titration type analytical procedures for the determination of kinetic parameters, such as, for instance, the stepwise titration method described in U.S. Patent Application Publication U.S. 2003/0143565 A1 and the “sequential kinetics methodology” described in U.S. patent application Ser. No. 10/861,098 (the disclosures of which are incorporated by reference herein), a ligand-supporting surface is sequentially contacted with different analyte solutions, e.g., stepwise changed analyte concentration, without intermediate regeneration or renewal of the immobilized ligand. In this case the response curve for the total experiment may be said to consist of a plurality of consecutive “binding curves”, one for each analyte solution (e.g., analyte concentration).

A basic feature of the invention is the automated assessment and selection of binding curves that are acceptable to be included in the final fit.

In one method variant, a cross-validation type procedure is used. Cross-validation, which is well known to the skilled person, is, for example, described in Wold S., Technometrics, 20 (1978) 397-406 (the relevant disclosure of which is incorporated by reference herein). The cross-validation may be performed either as a full cross-validation or a segmented cross-validation. In the first case, one binding curve is successively excluded at a time, and a fit is performed to the remaining curves and the result of the fit, e.g., expressed as the association rate constant or dissociation rate constant, is compared with that of the excluded curve. In this way unacceptable binding curves may be identified and excluded from the data set.

In segmented cross-validation, the data set is divided into a number of subsets, each of which are fitted separately and the results for each subset, e.g., expressed as the association rate constant or dissociation rate constant, are compared with each other. It is understood that this approach will reduce the number of necessary calculations to identify possible bad binding curves compared to a leave-one-out cross-validation.

In another method variant, a fit is made to the whole data set and the goodness of the fit with regard to each binding curve is then determined, e.g., by a residual analysis type procedure. This requires on the one hand, a descriptor for the goodness of the fit and, on the other hand, limits for the goodness defining if a binding curve is acceptable or not. Exemplary descriptors include, e.g., residual plots as mentioned above. Suitable limits may readily be determined by the skilled person. A final fit is then made after exclusion of the rejected curves.

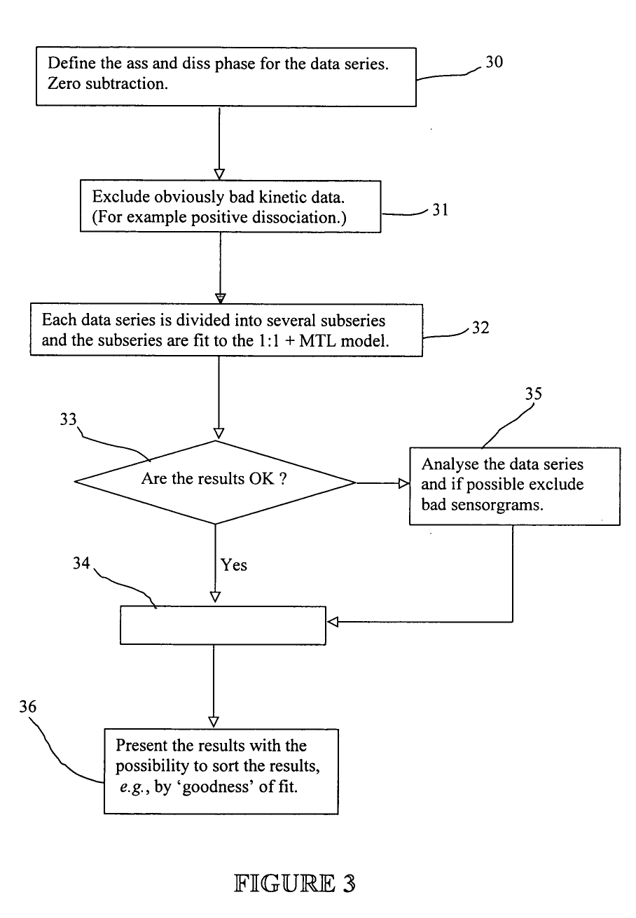

A (non-limiting) embodiment of the invention based on cross-validation will now be described with reference to the algorithm of FIG. 3. Assume that a kinetic analysis is to be made of binding data obtained for multiple analyte-ligand interactions, using, for example, an array (one- or two-dimensional) with a number of spots with different immobilized ligands and corresponding specific analytes to the ligands.

Preferably, a curve quality control is first performed to exclude sensorgrams with instrument-related defects (e.g., base-line slope, air spikes, carry-over between measurements), using the automated process described in the aforementioned U.S. Patent Application Publication U.S. 2004/0002167 A1 (the disclosure of which is incorporated by reference herein).

The particular analytes and immobilized ligand spots to be analysed are then selected by the operator, causing the relevant binding data for the kinetic analysis to be automatically extracted.

Referring now to FIG. 3, the first step (30) of the algorithm defines, for each data set or series (i.e., each group of sensorgrams corresponding to a particular analyte-ligand combination), the association and dissociation phases for the data series, or more particularly, the parts of the group of sensorgrams that are to be included in the analysis. Background noise is corrected for by subtracting a sensorgram describing a sample injection of a liquid with analyte concentration 0 (zero) from all sensorgrams describing a sample injection of a liquid with analyte concentration greater than 0 (zero). This procedure is referred to as zero subtraction.

In the next step (31), a simple quality control is performed by excluding curves with obviously erroneous kinetic data, such as, e.g., sensorgrams with a positive dissociation slope.

Then, in step (32), a cross-validation procedure is performed by dividing each data series, or group of sensorgrams, into several subseries or subgroups. Start guesses (kₐₛₛ, k_{diss}, Rₘₐₓ) are calculated for each subseries, and for each data series, the subseries are then fit to a kinetic model for the interaction, in the illustrated case 1:1 binding with mass transfer limitation (MTL).

The results of the fit from all subseries of a data series are put together (33). If there are only small differences between the different subseries, the results are considered to be acceptable, and a final fit is done by fitting the kinetic model to all accepted sensorgrams with start guesses taken from the cross-validation results (34).

If, on the other hand, there are large differences, a second quality control is performed by analysing the data series to find out if there is one or more sensorgrams that cause the bad result (35). If so, this or these sensorgrams are excluded and a final fit to the model is performed (34).

When this has been performed for all the data series (i.e., all combinations of analytes and immobilized ligands), the measuring results are presented (36) so that they may be sorted with regard to quality, e.g., by the “goodness” of fit, such as the above-mentioned chi-squared (chi2) or chi2/(Rₘₐₓ)². Optionally, several different goodness measures may be provided. The operator may now view all the fits and accept or reject results of the automatic evaluation performed.

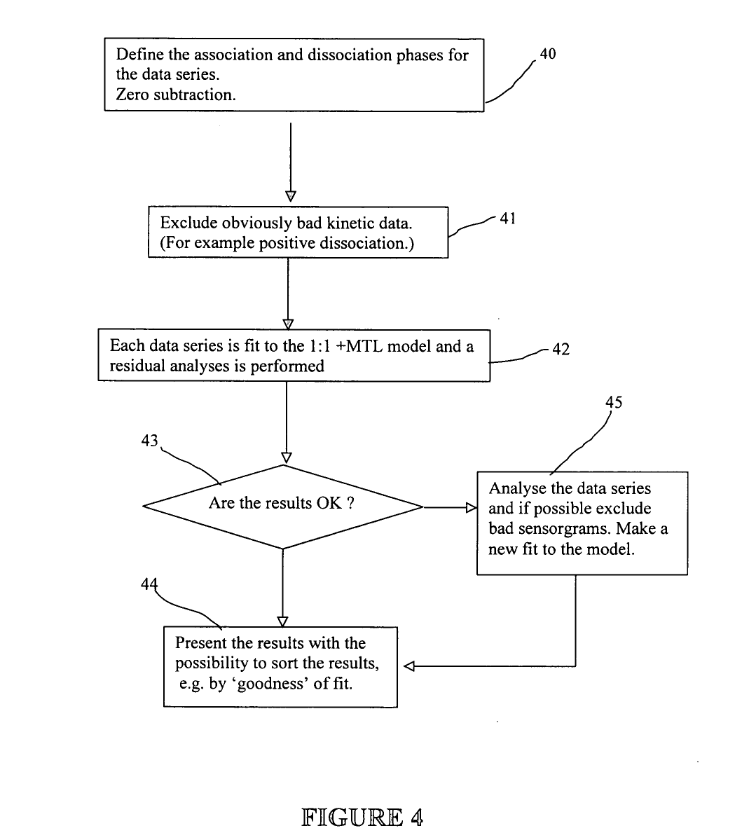

Another (non-limiting) embodiment of the invention based on residual analysis is described below with reference to FIG. 4.

In the same way as in the embodiment outlined in FIG. 3, the first step (40) of the algorithm defines the association and dissociation phases and makes a zero subtraction for each data series (each combination of analyte and ligand), and a simple quality control is performed in the second step (41).

In the next step (42), a global fit of each data series is made to a kinetic model for the interaction (here 1:1 binding with mass transfer limitation), and a residual analysis is made, i.e., using the kinetic parameters obtained in the global fitting. Fitted curves are produced for all sensorgrams, and the closeness of the fit to each curve is determined by residual values.

The residual values are then evaluated (43), and if all values are sufficiently small, i.e., below a predetermined level, the data series, and thereby the results of the fit, are accepted.

The quality of the fit, the reliability of the kinetic parameters and, optionally, other measures are determined, and the results are presented to the operator for examination and assessment (44).

If, on the contrary, the residual values are not acceptably small, the data series is analysed (45) to identify and exclude individual sensorgrams having too great residuals (outliers). It is understood that the exclusion criteria in this step (45) may be different from those used in step (43) above. A new fit to the kinetic model is then made on the modified data series.

Quality descriptors/measures are then determined and results are presented as described above (44).

After examination of the results presented in step (44), additional (bad) sensorgrams may optionally be excluded, and the modified data series be refitted, whereupon the final results may be presented.

The above-described procedure for automated determination of kinetic parameters, such as kinetic constants, is readily reduced to practice in the form of a computer system running software which implements the steps of the procedure. The invention also extends to computer programs, particularly computer programs on or in a carrier, adapted for putting the quality assessment procedure of the invention into practice. The carrier may be any entity or device capable of carrying the program. For example, the carrier may comprise a storage medium, such as a ROM, a CD ROM or a semiconductor ROM, or a magnetic recording medium, for example a floppy disc or a hard disk. The carrier may also be a transmissible carrier, such as an electrical or optical signal which may be conveyed via electrical or optical cable or by radio or other means. Alternatively, the carrier may be an integrated circuit in which the program is embedded.

While any suitable computer language may be used to implement the present invention, it is currently preferred to use a suite of MATLAB™ module files (The MathWorks, Inc., Natick, Mass., U.S.A.).

While the invention is generally applicable to the evaluation of kinetic data obtained in, e.g., real-time biointeraction analysis, an example of a particular application is for quality control in the production of protein drugs, i.e., for testing whether different batches of the same protein exhibit the same kinetics when binding to its target.

The invention will be further illustrated by the following non-limiting Example.

### EXAMPLE

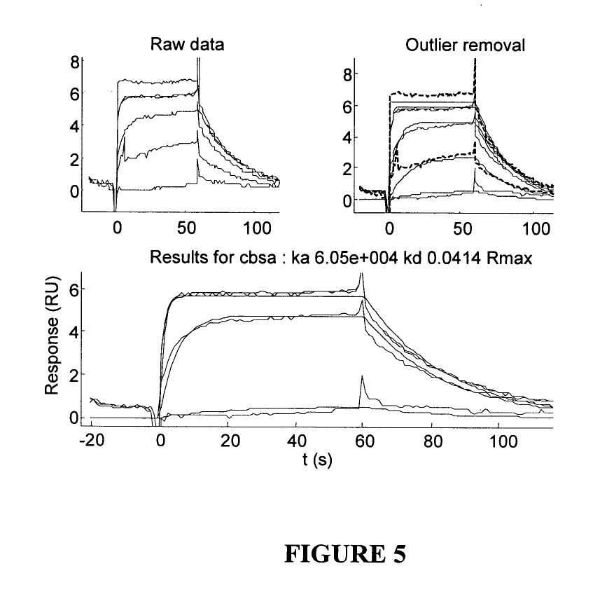

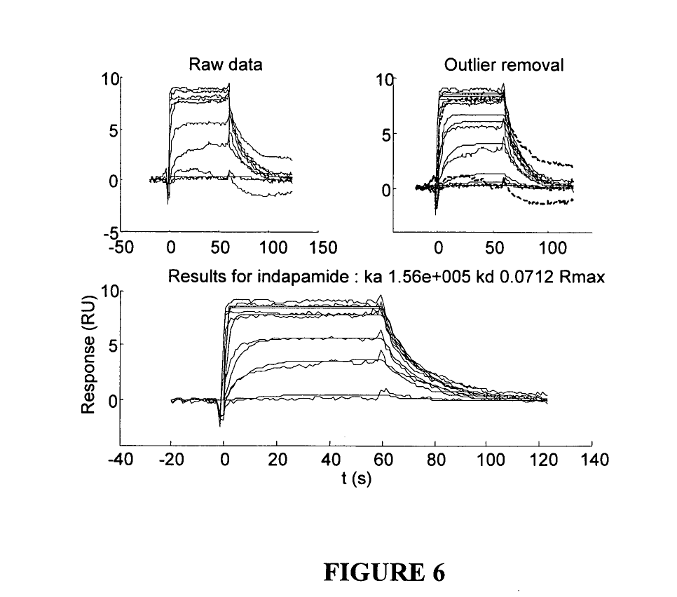

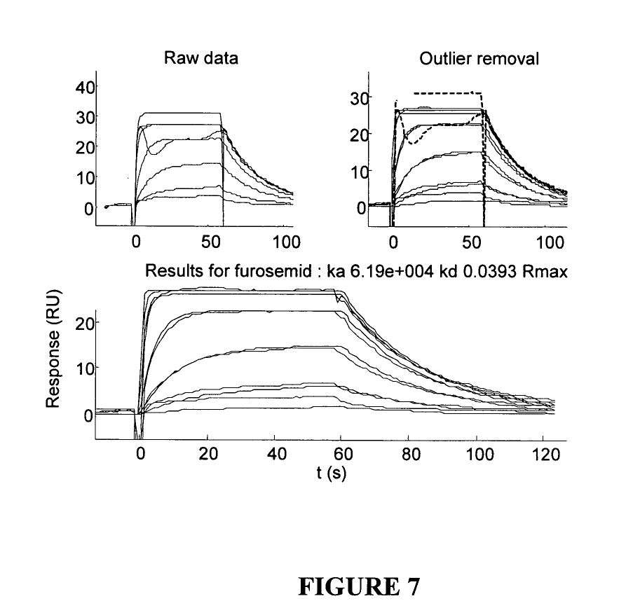

A BIACORE® S51 (Biacore AB, Uppsala, Sweden) was used to generate sensorgram raw data for the interaction of three drugs, CBSA (4-carboxybenzene-sulfonamide), indapamide and furosemide with carbonic anhydrase immobilized to Sensor Chip CM5 (Biacore AB, Uppsala, Sweden) (all reagents were from in-house sources, Biacore AB, Uppsala, Sweden). Each drug was injected at a number of different concentrations. The resulting sensorgram data are shown as sensorgram overlays “A” in FIGS. 5, 6 and 7, respectively.

The sensorgram raw data were then subjected to an automated kinetic evaluation for determining association rate constants, kₐ, and dissociation rate constants, k_{d}, by running a simple embodiment of the algorithm of the present invention in MATLAB 5.3.1.29215a (R11.1) (The MathWorks, Inc., Natick, Mass., U.S.A.), using a PC with Windows NT 4.0. The program used is shown below.

The results of the evaluation are shown in FIGS. 5, 6 and 7. At “B” in each figure are shown the sensorgram overlays shown at “A” but now supplemented with (i) the corresponding binding curves obtained by the curve fitting made by the program and shown in thin solid lines, and (ii) sensorgrams identified by the program as bad sensorgrams, or “outliers”, indicated by bold dashed lines. The resulting sensorgrams, and corresponding fitted binding curves, after exclusion of the outliers and a final fit performed by the program on the remaining sensorgrams, are shown at “C” in each figure. Also the kinetic constants for the different drugs are indicated in the respective FIGS. 5, 6 and 7.

It is to be understood that the invention is not limited to the particular embodiments of the invention described above, but the scope of the invention will be established by the appended claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

1. A computer-implemented method of determining at least one kinetic parameter for the interaction of an analyte in solution with an immobilized ligand from a data set comprising a plurality of different binding curves, each of which represents the progress of the interaction of the analyte with the ligand with time, which method comprises the steps of:
a) performing at least one fit of the whole data set or subsets thereof to a predetermined kinetic model for the interaction;
b) based on the result of the fit or fits performed in step a), identifying and excluding binding curves of unacceptable quality;
c) performing a final fit to the remaining data set; and
d) obtaining therefrom the kinetic parameter or parameters.

2. The method according to claim 1, wherein step c) is omitted and step d) is applied to the result of step a) when the remaining data set in step c) is identical to a data set to which a fit has been made in step a).

3. The method according to claim 2, wherein a fit is made to the whole data set in step a) of claim 1 and no binding curves are excluded in step b) of claim 1.

4. The method according to step 2, wherein fits are made to subsets of the whole data set in step a) of claim 1 and the remaining data set after exclusion of a binding curve or curves in step b) of claim 1 is identical to a data subset to which a fit has been made in step a) of claim 1.

5. The method according to claim 1, wherein a batch of data sets are processed, and wherein at least one kinetic parameter for each data set is determined.

6. The method according to claim 1, wherein step d) comprises presenting the results of the final fit sorted with regard to at least one quality parameter.

7. The method according to claim 6, wherein the quality parameter comprises goodness of the fit.

8. The method according to claim 1, wherein the exclusion of binding curves in step b) at least partly is based on residual analysis.

9. The method according to claim 1, wherein the exclusion of binding curves in step b) at least partly is based on cross-validation.

10. The method according to claim 9, wherein binding curves of unacceptable quality are identified by residual analysis.

11. The method according to claim 1, wherein at least one fit is made to the whole data set and the quality of the fit with respect to each binding curve is determined by residual analysis to identify binding curves of unacceptable quality.

12. The method according to claim 1, wherein the data set is divided into a plurality of subsets, a separate fit to the kinetic model is made for each subset, and the fits for the different subsets are compared with each other to determine if the data set contains binding curves of unacceptable quality.

13. The method according to claim 1, wherein steps a) and b) are repeated at least once before proceeding to step c).

14. The method according to claim 1, wherein step a) is preceded by a quality control to exclude binding curves which do not satisfy at least one predetermined curve quality criterion.

15. The method according to claim 1, wherein the kinetic model in step a) is a differential equation or a system of differential equations representing one to one binding with mass transfer.

16. The method according to claim 1, wherein the at least one kinetic parameter to be determined is selected from the association rate constant and the dissociation rate constant.

17. The method according to claim 1, wherein the plurality of binding curves included in each data set comprises binding curves representing different analyte concentrations.

18. The method according to claim 1, wherein the analyte-ligand interaction data of each data set is determined by a biosensor.

19. The method according to claim 18, wherein the biosensor is based on evanescent wave sensing.

20. The method according to claim 19, wherein the biosensor is based on surface plasmon resonance (SPR).

21. An analytical system for detecting molecular binding interactions, comprising:
(i) a sensor device comprising at least one sensing surface, detection means for detecting molecular interactions at the at least one sensing surface, and means for producing detection data representing binding curves which represent the progress of each interaction with time, and
(ii) data processing means for performing steps a) to d) of claim 1.

22. A computer program comprising program code means for performing the kinetic parameter determination of claim 1 when the program is run on a computer.

23. A computer program product comprising program code means stored on a computer readable medium or carried on an electrical or optical signal for performing the kinetic parameter determination of claim 1 when the program is run on a computer.

24. A computer system containing a program for performing the kinetic parameter determination of claim 1.
